SEQUENCE LISTING (14 sequences)

SEQ ID NO: 1 - DNA, 36 bp, Homo sapiens
gccaccctcgggtgaaagtgggcaatgagtacgtca

SEQ ID NO: 2 - DNA, 37 bp, Homo sapiens
tgccaccctcaggtgaaagtgggcaatgagtacgtca

SEQ ID NO: 3 - protein, 30 aa, Homo sapiens
IMHFGNMKFKLKQREEQAEPDGTEEADKSA

SEQ ID NO: 4 - DNA, 90 bp, Homo sapiens
ctacctcatggggctgaactcagccgacctgctcaaggggctgtgccaccctcgggtgaaagtgggcaatgagtacgtcaccaaggggca

SEQ ID NO: 5 - DNA, 90 bp, Homo sapiens
gatggagtaccccgacttgagtcggctggacgagttccccgacacggtgggagcccactttcacccgttactcatgcagtggttccccgt

SEQ ID NO: 6 - protein, 30 aa, Homo sapiens
YLMGLNSADLLKGLCHPRVKVGNEYVTKGQ

SEQ ID NO: 7 - protein, 29 aa, Homo sapiens
RMPSFEASRSLPSHWGRTFTPLSYTVLPC

SEQ ID NO: 8 - DNA, 36 bp, Homo sapiens
gccaccctcaggtgaaagtgggcaatgagtacgtca

SEQ ID NO: 9 - DNA, 105 bp, Homo sapiens
aggggctgtgccaccctcgggtgaaagtgggcaatgagtacgtcaccaaggggcagaatgtccagcaggtgggtccatcttcagatgataatgggtgggcagggt

SEQ ID NO: 10 - protein, 34 aa, Homo sapiens
GLCHPRVKVGNEYVTKGQNVQQVGPSSDDNGWAG

SEQ ID NO: 11 - DNA, 105 bp, Homo sapiens
aggggctgtgccaccctcaggtgaaagtgggcaatgagtacgtcaccaaggggcagaatgtccagcaggtgggtccatcttcagatgataatgggtgggcagggt

SEQ ID NO: 12 - protein, 34 aa, Homo sapiens
GLCHPQVKVGNEYVTKGQNVQQVGPSSDDNGWAG

SEQ ID NO: 13 - protein, 48 aa, Homo sapiens
EEADKSAYLMGLNSADLLKGLCHPRVKVGNEYVTKGQNVQQVIYATGA

SEQ ID NO: 14 - protein, 48 aa, Homo sapiens
EEADKSAYLMGLNSADLLKGLCHPQVKVGNEYVTKGQNVQQVIYATGA

(19) United States
(12) Patent Application Publication
(10) Pub. No.: US 2019/0185816 A1
(43) Pub. Date: Jun. 20, 2019

(54) CARDIAC MICROTISSUE AND USES THEREOF

(71) Applicant: THE REGENTS OF THE UNIVERSITY OF MICHIGAN, Ann Arbor, MI (US)

(72) Inventors: Todd Herron, South Lyon, MI (US); Jianping Fu, Ann Arbor, MI (US)

(21) Appl. No.: 16/225,857

(22) Filed: Dec. 19, 2018

Related U.S. Application Data
(60) Provisional application No. 62/607,511, filed on Dec. 19, 2017.

(51) Int. Cl. C12N 5/077; C12N 5/00; C12N 5/074; C12M 3/00; C12M 1/12

(52) U.S. Cl. CPC C12N 5/0657; C12N 5/0062; C12N 2533/54; C12M 21/08; C12M 25/14; C12N 5/0696

(57) ABSTRACT

The present disclosure provides compositions and methods employing stem cell-derived cardiomyocytes. In some embodiments, compositions for assessing cardiotoxicity using 3-dimensional cultures of stem-cell cardiomyocytes are provided.

This application claims the benefit of U.S. provisional application Ser. No. 62/607,511, filed Dec. 19, 2017, which is incorporated herein by reference in its entirety.

### FIELD

The present disclosure provides compositions and methods employing stem cell-derived cardiomyocytes. In some embodiments, three dimensional cardiac tissues and uses thereof are provided.

### BACKGROUND

Stem cells are pluripotent cells with remarkable potential to develop into many different cell types in the body during early life and growth. In addition, in many tissues they serve as a sort of internal repair system, dividing essentially without limit to replenish other cells as long as the person or animal is still alive. There are two types of stem cells: embryonic stem cells and non-embryonic “somatic” or “adult” stem cells. Induced pluripotent stem cells (iPSCs) are adult cells that have been genetically reprogrammed to pluripotent stem cells.

Stem cells carry promises for regenerative medicine and cell therapy, but are also changing the drug discovery and development process. Emergence of stem cell technologies provides new opportunities to build innovative cellular models. Stem cell models offer new opportunities to improve the manner in which pharmaceutical companies identify lead candidates and bring new drugs to the market. In spite of promising applications, new competencies surrounding stem cell differentiation and proliferation, induction of pluripotent stem cells and creation of efficacy assays are needed to make successful use of stem cells in drug discovery.

Beyond improved models, pluripotent stem cells technologies are introducing applications that were previously not possible. Currently, human clinical populations are poorly represented in drug development with a lack of genetic heterogeneity in human cellular models and a limited number of human disease models. As a result of induced pluripotent stem cell (iPSC) technology, new cellular models can be created from individuals with a diverse range of drug susceptibilities and resistances, offering the promise of a “clinical trial in a dish” in a field where a personalized medicine approach is becoming increasingly predominant.

Despite these advantages there are still several challenges in using stem cells in drug discovery. The differentiation and reprogramming strategies are not standardized and are often based on growth factors, making protocols expensive, poorly reproducible and limited in terms of scale-up. The pace of stem cell research—for example, a single differentiation or reprogramming experiment currently can take more than a month—is too slow to fit into timelines required by the industry. In addition, before pharmaceutical companies typically will invest in the development of such platforms, further demonstrations of success and potential applications are necessary. And last but not least, stem cell culture and differentiation need to be adapted to the high-throughput environment of drug discovery by developing standardized high-throughput and miniaturized assays for in vitro screening.

### SUMMARY

The present disclosure provides compositions and methods employing stem cell-derived cardiomyocytes. In some embodiments, three dimensional cardiac tissues and uses thereof are provided.

The present disclosure provides three dimensional cardiac microtissues. The microtissues more closely mimic native cardiac tissue relative to monolayer or two dimensional culture of cells. The microtissues find use in research (e.g., disease models), screening (e.g., drug screening), and therapeutic applications.

For example, in some embodiments, provided herein is a method of generating a three dimensional cardiac microtissue, comprising: a) differentiating induced pluripotent stem cells (iPSCs) to a population of cells comprising cardiac myocytes; b) contacting the population of cardiac myocytes with a solution comprising, consisting essentially of, or consisting of collagen; and c) transferring the collagen solution comprising cardiac myocytes into a solid support and culturing under conditions such that the myocytes form a three-dimensional cardiac microtissue. In some embodiments, the solid support comprises a plurality of microtissue culturing molds each comprising a plurality of posts spaced at a uniform distance. In some embodiments, each of the microtissue molds comprises two posts. In some embodiments, the posts are 0.5 to 5 mm (e.g., 1 to 3 mm or 2 mm) apart. In some embodiments, the posts are 0.2 to 1 mm (e.g., 0.5 mm) in diameter. In some embodiments, the microtissue molds are approximately 2×4×1.5 mm. In some embodiments, the microtissue molds and posts are made of polydimethylsiloxane (PDMS). In some embodiments, the three-dimensional microtissue forms suspended between said posts. In some embodiments, the iPSCs comprise an Arg403Gln mutation in the myosin heavy chain 7 (MYH7; GenBank Accession number NP_000248 XP_005267753) gene. In some embodiments, the three-dimensional microtissue comprising an Arg403Gln mutation exhibit one or more properties of hypertrophic cardiomyopathy (HCM) (e.g., including but not limited to, structural disorganization, intracellular sarcomere disarray, reduced contractile force, arrhythmia, or electro-mechanical dysfunction). In some embodiments, the population of cells comprises approximately 75% cardiac myocytes. In some embodiments, the three dimensional cardiac microtissue exhibits beats. In some embodiments, the three dimensional cardiac microtissue exhibits one or more parameters selected from electrophysiological maturation or contraction.

Further embodiments provide a method of generating a three dimensional cardiac microtissue exhibiting properties of HCM, comprising: a) differentiating induced pluripotent stem cells (iPSCs) comprising a MYH7 Arg403Gln mutation to a population of cells comprising cardiac myocytes; b) contacting the population of cardiac myocytes with a collagen solution; and c) transferring the collagen solution comprising cardiac myocytes into or onto a solid support and culturing under conditions such that said myocytes form a three-dimensional cardiac microtissue exhibiting properties of HCM.

Additional embodiments provide a three dimensional cardiac microtissue generated by the methods described herein. In some embodiments, the microtissue is suspended between two posts of a solid support comprising a plurality of microtissue culturing molds each comprising a plurality of posts spaced at a uniform distance.

Yet other embodiments provide a system, comprising: a) a population of cells comprising cardiac myocytes; and b) a solid support comprising a plurality of microtissue culturing molds each comprising a plurality of posts spaced at a uniform distance.

Still further embodiments provide a method of screening compounds, comprising: a) contacting a three dimensional cardiac microtissue described herein with a test compound; and b) assaying the effect of the test compound on one or more properties of the cardiac microtissue. In some embodiments, the test compound is a candidate drug to treat cardiac myopathy. In some embodiments, the test compound is any compound in which cardiac toxicity is a concern (e.g., assessment of QT prolongation).

In some embodiments, provided herein is a composition, comprising: a solid support comprising a plurality of microtissue culturing molds each comprising a plurality of posts spaced at a uniform distance, wherein a three dimensional cardiac microtissue is suspended between two of the posts. In some embodiments, the composition is provided as part of a system comprising one or more test compounds.

Certain embodiments provide a method of assaying cardiac tissue self-assembly, comprising: a) contacting a population of cardiac myocytes with a collagen solution; b) transferring the collagen solution comprising cardiac myocytes into a solid support and culturing under conditions such that the myocytes form a three-dimensional cardiac microtissue; and c) assaying the formation of the three-dimensional cardiac microtissue. In some embodiments, the assaying comprises quantitating the kinetics of formation and/or observing the development of abnormalities in the three-dimensional cardiac microtissue. In some embodiments, the cardiac myocytes comprise a genetic variation or mutation. In some embodiments, the method further comprises the step of contacting the cardiac myocytes with a drug or toxin prior to and/or during the formation of three-dimensional cardiac microtissue.

Further embodiments are described herein.

### DESCRIPTION OF THE FIGURES

FIG. 1 shows HCM Stem Cell Line Characterization. A. Confirmation of successful reprogramming of HCM patient specific skin cells to stem cells by staining for stem cell specific pluripotent markers TRA-1-60, TRA-1-81 and Oct3/4. B. Normal karyotype of HCM stem cells. C. 20× bright field images of control and HCM myocytes showing a clear difference in cell size (3339.8±35.1 μm² vs. 84614.1±318.3 μm², n=702, 1219, p<0.05, scale bars 80 μm). D. Immunostaining of control (19-9-11) and HCM, showing extensive hypertrophy, decreased Cx43 and sarcomere disorganization in HCM myocytes. E. Western blot showing GAPDH and pro-BNP of control and HCM purified myocytes and quantification normalized to GAPDH and control (1.0±0.3 vs. 8.9±1.7, n=3, 3, p<0.05).

FIG. 2 shows 3D Microtissues. A. Top and side view of 3D microtissue mold with microtissues. B. A 3D microtissue's displacement during contraction with corresponding calcium transient and optical motion traces.

FIG. 3 shows immunostaining of 3D Microtissues. A-B. Cell membrane stain WGA-AF488 reveals elongated and aligned myocytes around microtissue posts with heterogeneity of alignment between posts. C. cTnT and DAPI immunostaining of micro-tissue removed from post showing myocyte distribution throughout the micro-tissue. D. α-actinin and DAPI immunostaining showing alignment of both myocytes and sarcomeres and elongation of nuclei.

FIG. 4 shows HCM 3D Microtissue Formation Kinetics and Cellular Organization. A. Comparison of control and HCM 3D microtissue formation over 84 hrs. 3D self-assembly over 84 hours, HCM (diamonds) decreasing to 2.89±0.31 mm², 62±4.1% (n=11) of their initial size while controls (squares) reform to be 1.70±0.17 mm², a 77.7±2.2% reduction, (n=39, p<0.05 at each time point >0.5 hrs, images are each 2×4 mm). B. Images of CDI HCM and CDI isogenic control tissues over 48 hours of formation (scale 3.5 mm).

FIG. 5 shows electrophysiology of 3D Microtissues. HCM microtissues have significant structural and electrophysiological alterations. A-B. Example control and HCM microtissues on day 7 after plating with corresponding spontaneous calcium transients (dashed) and optical motion traces (solid). C. HCM tissue width is significantly larger than controls; 0.63±0.1 vs. 0.92±0.5 mm (n=38, 25). D. Average spontaneous frequencies are not different between groups; 0.60±0.04 Hz vs. 0.62±0.05 Hz (p=0.85). E. Calcium transient amplitude (CaTA) is significantly greater in control traces; 0.59±0.06 vs. 0.21±0.02. F. Control microtissues generate about 3 times the force of HCM microtissues; 680.1±112.4 vs. 209.5±44.5 μN. G. HCM CaTD₈₀ is significantly greater than controls 887.9±20.8 vs. 1042.0±53.1 (p<0.05). H. As is the time to peak; 274.5±9.2 vs. 404.8±24.8 ms, (control n=38, HCM n=25, p<0.05).

FIG. 6 shows generation of an HCM Adenoviral Vector. A. Western Blot of β-MyHC and total myosin shows progressive daily overexpression of the wild type ad-WT-MYH7 and HCM ad-Arg403Gln-MYH7 in adult rat cells. B. Immunostaining of β-MyHC shows that the overexpression of ad-Arg403Gln-MYH7 results in appropriate sarcomere incorporation. C. Immunostaining of 2D plated 19-9-11 control hiPSC-CMs showing how the overexpression of the Arg403Gln HCM mutated myosin results in marked hypertrophy and sarcomere disorganization and decreased Cx43 expression in comparison to untreated myocytes.

FIG. 7 shows 3D Acute Gene Transfer of Ad-Arg403Gln-MYH7 HCM Mutation. A. Examples of an uninfected control, ad-WT-MYH7 and ad-Arg403Gln-MYH7 expressing microtissues. B. Quantification of microtissue width: both control and WT expressing tissues were significantly smaller than the HCM Arg403Gln expressing. (Control: 658.45±13.1 μm, WT: 640.6±5.3 μm, Arg403Gln: 718.1±8.0 μm, n=25, 52, 50, p<0.05). C. Acute gene transfer of the patient's HCM mutation into both control lines resulted in hypertrophy, but not to the same extent as the HCM patient's microtissues.

FIG. 8 shows arrhythmias. A. Representative traces of an HCM and Control arrhythmia. B. Arrhythmic propensity of HCM (top) and Control (bottom) microtissues. C. Arrhythmic HCM tissues (stripes) were significantly larger than HCM tissues with regular activity (solid), while no difference in size was observed in controls with or without arrhythmias; 0.92±0.24 vs. 1.13±0.38 mm for HCM (n=25, 21, p<0.05) and 0.63±0.01 vs. 0.63±0.05 mm in Controls (n=38, 4, not significant).

FIG. 9 shows familial HCM patient pedigree.

FIG. 10 shows sequencing of the HCM β-Myosin heavy chain mutation Arg403Gln-MYH7. A. Sequencing of the β-myosin heavy chain for the control (19-9-11) and both HCM lines (HCM and HCM (E1)), the wild type β-myosin heavy chain amino acid sequence is shown above the HCM Arg403Gln point mutation. B. Sequence of hiPS isogenic control (01178.103) and hiPS HCM R403Q (01178.103) genomic DNA. C. Site directed mutagenesis of HCM β-myosin heavy chain mutation Arg403Gln-MYH7.

FIG. 11 shows myocyte density. A. Examples of the percentage of cardiac troponin T (cTnT) positive cells in control and HCM myocyte differentiations as determined by flow cytometry, myocyte percentages were similar in control and HCM tissues (74.5±4.1% vs. 72.4±3.2%, n=3, 4). B-D. Flow cytometry adjusting the ratio of myocytes (cTnT+) and non-myocytes (cTnT−) with magnetic purification. E. Schematic of 3D microtissue formation showing the side and top views (cells and collagen mixture green, PDMS mold brown). F. Non-myocytes were necessary for 3D microtissue formation.

FIG. 12 shows dual optical mapping of 3D microtissues as a high throughput technique.

FIG. 13 shows measuring the spring constant. A-B. Using a force transducer on a 10:1 (w/w) PDMS to curing agent post of a height of 1.5 mm and radius of 250 μm, the force-distance relationship to spring constant (k) from the slope was plotted. C. Top: Schematic showing the height of the microtissue at rest, where the height of the post is L, height of the tissue is a. Bottom: Shows the posts as the tissue contracts and the displacement (x). D. Hooke's Law: having calculated the elastic modulus of our posts and along with the post dimensions and measuring the height of the tissues on the posts, the spring constant of the microtissues system was determined, k=6.468.

FIG. 14 shows quantification of cell size. A. Schematic of high throughput processing, 216 images can be obtained from a 6 well dish of cells for analysis. B. Examples of high volume processing definition created to include only single cells to determine average cell size (included single cells are masked in gold, scale bar 200 μm). C. Immunostaining of hiPSC-CMs for DAPI and α-actinin showing good sarcomere organization in controls (BJ) but not HCM myocytes.

FIG. 15 shows simultaneous voltage and calcium optical recordings. Dual traces from a 2D monolayer (A) and 3D micro-tissue (B) each paced at 1 Hz show a shortening of both action potential duration (APD) and calcium transient duration (CaTD) in the 3D micro-tissues. C-D. 3D microtissues had a significant APD₈₀ and CaTD₈₀ shortening at 0.5, 1 and 1.5 Hz (APD₈₀: (0.5 Hz) 549.5±26.9 ms vs. 676.8±30.8 ms, (1 Hz) 395.0±8.7 ms vs. 526.9±22.2 ms, (1.5 Hz) 375.6±7.7 ms vs. 414.3±15.1 ms, CaTD₈₀: (0.5 Hz) 735.4±13.2 ms vs.957.5±35.9 ms, (1 Hz) 580.9±8.6 ms vs. 681.8±15.2 ms, (1.5 Hz) 454.2±3.8 ms vs. 505.9±8.2 ms, n=8, 10, 9; n=12, 18, 12, *p<0.05 for each frequency).

FIG. 16 shows hypertrophic pro-BNP response. A-B. Pro-BNP dose response for control and HCM myocytes (*p<0.05 ANOVA multiple comparisons Bonferroni correction). C. Comparison of the change in Pro-BNP between control and HCM treated myocytes (n=3, 3, student T-test with Welch correction).

FIG. 17 shows hypertrophic pro-BNP response staining. A. Staining of pro-BNP on control and HCM cells at different endothelin-1 concentrations. B. Staining of pro-BNP on control and HCM cells at different endothelin-1 concentrations imaged by the IncuCyte used for fluorescence quantification.

FIG. 18 shows CDI myocytes: hypertrophic pro-BNP response. A. Western blots showing the pro-BNP response for CDI control and CDI HCM myocytes with different amounts of ET-1. B. Staining of pro-BNP on control and HCM cells at different endothelin-1 concentrations imaged by the IncuCyte used for fluorescence quantification.

FIG. 19 shows 3D microtissue drug response. 3D microtissues respond to typical pharmacological treatments as expected. A-B. I_{Kr} blocker E-4031 significantly lengthened the action potential duration (APD₈₀) of both control and HCM 3D microtissues. C-D. 3D microtissues responded to Isoproterenol (I_{SO}) by significantly increasing the spontaneous frequency in both controls and HCM tissues and shortening the calcium transient duration (CaTD₈₀). (* indicates a difference between controls and HCM tissues, ‡ indicates significance between controls before and after treatment, while † indicates significance for HCM before and after treatment, p<0.05).

FIG. 20 shows response of 3D HCM microtissues to exposure to isoproterenol (I_{SO}) and I_{Kr} blocker E-4031.

### DEFINITIONS

To facilitate an understanding of the present technology, a number of terms and phrases are defined below. Additional definitions are set forth throughout the detailed description.

As used herein, the term “treatment” is defined as the application or administration of a therapeutic agent described herein (e.g., composition comprising cardiac microtissues) to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

As used herein, the term “functionally mature cardiomyocytes” refers to cardiomyocytes or cardiac microtissues (e.g., prepared using the methods described herein) that exhibit one or more properties of primary cardiomyocytes or heart muscle (e.g., electrophysiological properties described herein). In some embodiments, “functionally mature cardiomyocytes” are also referred to as “electrophysiologically mature cardiomyocytes.”

By “pharmaceutically acceptable” is meant that the ingredients of the pharmaceutical composition are compatible with each other and not deleterious to the recipient thereof.

The term “subject” as used herein refers to an animal, preferably a mammal, most preferably a human, who has been the object of treatment, observation, or experiment.

The term “effective amount” as used herein means that amount of an agent (e.g., cardiac microtissue) that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptoms of the disease or condition being prevented.

“Feeder cells” or “feeders” are terms used to describe cells of one type that are co-cultured with cells of another type, to provide an environment in which the cells of the second type can grow. When a cell line spontaneously differentiates in the same culture into multiple cell types, the different cell types are not considered to act as feeder cells for each other within the meaning of this definition, even though they may interact in a supportive fashion. “Without feeder cells” refers to processes whereby cells are cultured without the use of feeder cells.

A cell is said to be “genetically altered” when a polynucleotide has been transferred into the cell by any suitable means of artificial manipulation, or where the cell is a progeny of the originally altered cell that has inherited the polynucleotide. The polynucleotide will often comprise a sequence encoding a protein of interest, which enables the cell to express the protein at an elevated level. The genetic alteration is said to be “inheritable” if progeny of the altered cell have the same alteration.

### DETAILED DESCRIPTION

The present disclosure provides compositions and methods employing stem cell-derived cardiomyocytes. In some embodiments, three dimensional cardiac tissues and uses thereof are provided. Exemplary methods of generating and using three dimensional cardiac tissues are described herein.

### I. Generation of Three Dimensional Cardiac Tissues

In some embodiments, three dimensional tissue are generated from cardiac myocytes. In some embodiments, the myocytes are generated by differentiating pluripotent cells (e.g. induced pluripotent stem cells (iPSCs).

### Cells

A wide variety of cells and stem cells may be employed with the technology described herein. In some embodiments, the cell is a pluripotent cell with potential for cardiomyocyte differentiation. Such cells include embryonic stem cells and induced pluripotent stem cells, regardless of source. For example, induced pluripotent stem cells may be derived from stem cells or adult somatic cells that have undergone a dedifferentiation process (e.g., a donor).

Induced pluripotent stem cells may be generated using any known approach. In some embodiments, iPSCs are obtained from adult human cells (e.g., fibroblasts). In some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, l-myc), Nanog, LIN28, Glis1, etc.) or mimicking their activities is employed to generate iPSCs (using transgenic vector (adenovirus, lentivirus, plasmids, transposons, etc.), inhibitors, delivery of proteins, microRNAs, etc.).

In some embodiments the cells are non-terminally differentiated cells (regardless of pluripotency) or other non-maturated cells.

In some embodiments, cells are screened for propensity to develop teratomas or other tumors (e.g., by identifying genetic lesions associated with a neoplastic potential). Such cells, if identified, may discarded.

### Culturing/Differentiation

In some embodiments, cardiac myocytes (cardiomyocytes) are generated by differentiation of pluripotent cells. Pluripotent (e.g., stem) cells are cultured and/or differentiated on a flexible (e.g., soft, pliable) surface coated with ECM proteins. In some embodiments, the culture platform is selected based on its ability to produce cells of equivalent quality as those generated with the matrigel on PDMS of the experimental examples described below. As such, the flexibility of the surface is such that cells having one or more of the desired properties herein are generated. Likewise the constituents of the ECM are selected to achieve the same result.

Culture conditions are selected based on the cells employed. In some embodiments, the conditions used are those of Lee et al., 2012 or references 30 and 31. In some embodiments, the process comprises thawing (if cryopreserved) and plating iPSCs on the coated support at a desired density (e.g., 125,000 cells per monolayer) in differentiation media (e.g., embryoid body differentiation media, commonly referred to as embryoid body-20, comprising 80% Dulbecco Modified Eagle Medium (DMEM/F12), 0.1 mmol/L⁻¹ nonessential amino acids, 1 mmol/L⁻¹ L-glutamine, 0.1 mmol/L⁻¹ β-mercaptoethanol, and 20% fetal bovine serum; Gibco) supplemented with 10 μmol/L⁻¹ blebbistatin. After 24 hours in embryoid body-20, the medium is switched to iCell maintenance medium (Cellular Dynamics), supplemented with 10 μmol/L⁻¹ blebbistatin, and cells are cultured for an additional time period (e.g., 96 hours) at 37° C., in 5% CO₂, with the medium changed once daily.

### Generation of Three Dimensional Tissues

In some embodiments, populations of cardiomyocytes are used to generate three dimensional cardiac tissues. In some embodiments, the method described in Example 1 is utilized. For example, in some embodiments, the method comprises contacting the population of cardiac myocytes with a collagen solution; and transferring the collagen solution comprising cardiac myocytes into or onto a solid support and culturing under conditions such that the myocytes form a three-dimensional cardiac microtissue.

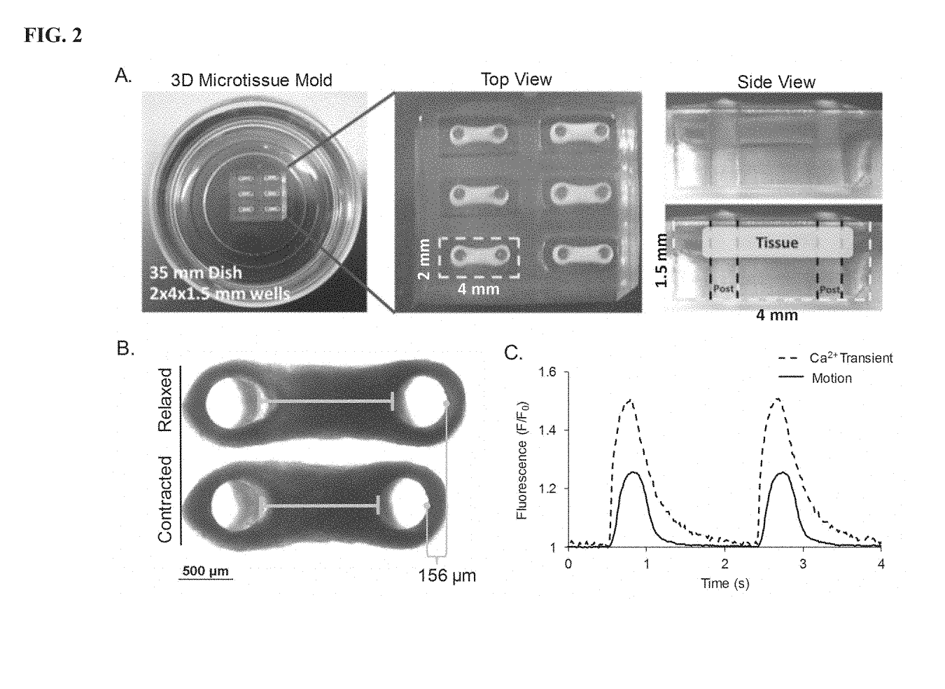

Exemplary solid supports are described in FIG. 2. In some embodiments, each solid support comprises a plurality of microtissue culturing molds. In some embodiments, the molds are cast (e.g., from PDMS or other suitable material). As shown in FIG. 2a, each of the microtissue culturing molds comprises a plurality of posts spaced at a uniform distance. In some embodiments, each of the microtissue molds comprises two posts. In some embodiments, the posts are 0.5 to 5 mm (e.g., 1 to 3 mm or 2 mm) apart. In some embodiments, the posts are 0.2 to 1 mm (e.g., 0.5 mm) in diameter. In some embodiments, the microtissue molds are approximately 2×4×1.5 mm.

In use, the collagen solution comprising cardiomyocytes is placed in the mold. In some embodiments, the population of cells comprises approximately 75% (e.g., 60-90% or 90-100%) cardiac myocytes, with the remainder of cells non-myocyte cells. The solution is then cultured until collagen is crosslinked (e.g., spontaneously) and three dimensional microtissues are formed. In some embodiments, the three-dimensional microtissue forms suspended between the posts of the mold. As described herein, cultured tissues suspended between posts leads to improved electrophysiological and contraction properties. At this point, tissues are removed and cultured, if needed, before use.

The resulting three dimensional tissues exhibit improved properties relative to two dimensional cultures that more closely mimic in vivo cardiomyocytes. For example, in some embodiments, the three dimensional cardiac microtissue exhibits beats. In some embodiments, the three dimensional cardiac microtissue exhibits one or more parameters selected from electrophysiological maturation or contraction.

In some embodiments, variant tissues that mimic diseases are generated. For example, in some embodiments, mutations (e.g., Arg403Gln) that generate tissues with hypertrophic cardiomyopathy (HCM) properties are utilized. In some embodiments, the three-dimensional microtissue comprising an Arg403Gln mutation exhibit one or more properties of hypertrophic cardiomyopathy (HCM) (e.g., including but not limited to, structural disorganization, intracellular sarcomere disarray, reduced contractile force, arrhythmia, or electro-mechanical dysfunction). HCM is an inherited autosomal dominant condition that affects 1 in 500 people (Maron B J. Jama. 2002; 287:1308-1320). It is most often genetically defined by mutations in sarcomeric proteins such as the β-myosin heavy chain (β-MyHC) and myosin binding protein C (Maron 2002, supra; Tardiff J C. Heart Fail Rev. 2005; 10:237-248), while the physiological hallmarks of the HCM phenotype include ventricular wall thickening, cellular hypertrophy and myocyte disarray (Geisterfer-Lowrance A A, et al., Cell. 1990; 62:999-1006; Lopes L R, et al., Heart. 2013; 99:1800-1811). Although numerous HCM mutations have been identified, it is still not well understood how missense mutations of contractile protein genes result in the structural and electrical defects observed in cardiac cells or how they lead to heart failure, stroke and sudden death (Maron B J, et al., Circulation. 1995; 92:785-789; Kumar K R, et al., Card Electrophysiol Clin. 2015; 7:173-186; Lankford E B, et al., J Clin Invest. 1995; 95:1409-1414).

### II. Uses

Three dimensional cardiac microtissues provided herein find use in a variety of research, diagnostic, screening, and therapeutic applications.

In some embodiments, the tissues are used for disease modeling and drug development (e.g., HCM). The quality of the tissues and the ability to generate them in a short period of time makes them ideally suited for such research uses, particularly high-throughput analysis. Agents (e.g., antiarrhythmic or other candidate HCM agents) are contacted with the tissues to determine the effect of the agent (e.g., on one or more electrophysiological or contractile properties described herein). Tissues may also be modified to include a marker and used either in vitro or in vivo as diagnostic compositions to assess properties of the tissues in response to changes in the in vitro or in vivo environment.

Tissues also find use in therapeutic approaches, including, but not limited to, transplantation of the cells into a subject (e.g., for tissue repair, to prevent or treat a disease or condition) or organ synthesis.

In some embodiments, tissues are used in drug testing applications. For example, in some embodiments, drugs or biological agents are tested. Indications for drug testing include any compound or biological agent in the pharmaceutical discovery and development stages, or drugs approved by drug regulatory agencies, like the US Federal Drug Agency. All classes of drugs, ethical, over-the-counter and nutraceuticals for any medical indications, such as but not limited to, drugs for treating cancer, neurological disorders, fertility, vaccines, blood pressure, blood clotting, immunological disorders, anti-infectives, anti-fungals, anti-allergens, and cardiovascular related disorders.

In some embodiments, drug testing applications determine the effects of new chemical entities on cardiac electrophysiological function and contractile properties including, but not limited to, action potential duration, beating frequency, calcium transient duration (CaTD), contractile properties and arrhythmia characterization. Such assays serve to inform drug development businesses on the risk of a compound to cause fatal cardiac arrhythmia or other heart-related side effects. These tests may be acute or performed following long term exposure to a drug.

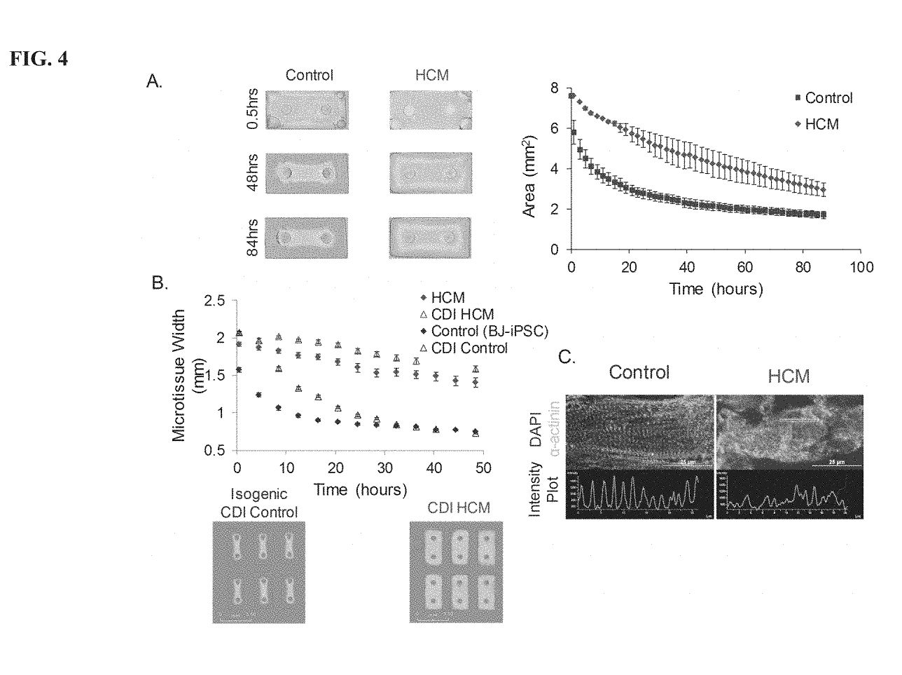

The tissues described herein further find use in the study of congenital heart defects. In some embodiments, as shown in FIG. 4, quantification of cardiac organogenesis is performed to study heart formation. In some embodiments, the effect of genetic or environmental factors (e.g., toxins or drugs) on heart formation is assayed by simulating heart development that occurs in utero (e.g., using the tissues described herein). Quantification of the kinetics of self-assembly as shown in FIG. 4 simulates anatomical formation of the 3D structure of the heart. Thus, suspected fetal toxins can be assayed in vitro using the tissues described herein. In some embodiments, drug testing and research applications are performed on tissues in the culturing molds described herein.

Embodiments of the present disclosure provide kits comprising the tissues described herein. For example, in some embodiments, kits comprise cells (e.g., cardiomyocytes or iPSC or stem cells suitable for differentiating into cardiomyocytes) in or on a culturing mold (e.g., as described herein). In some embodiments, kits further comprise reagents for differentiation or use of cells (e.g., buffers, test compounds, controls, etc.).

### EXAMPLES

Unless specified otherwise, the following experimental techniques were used in the Examples.

### Example 1

### Methods

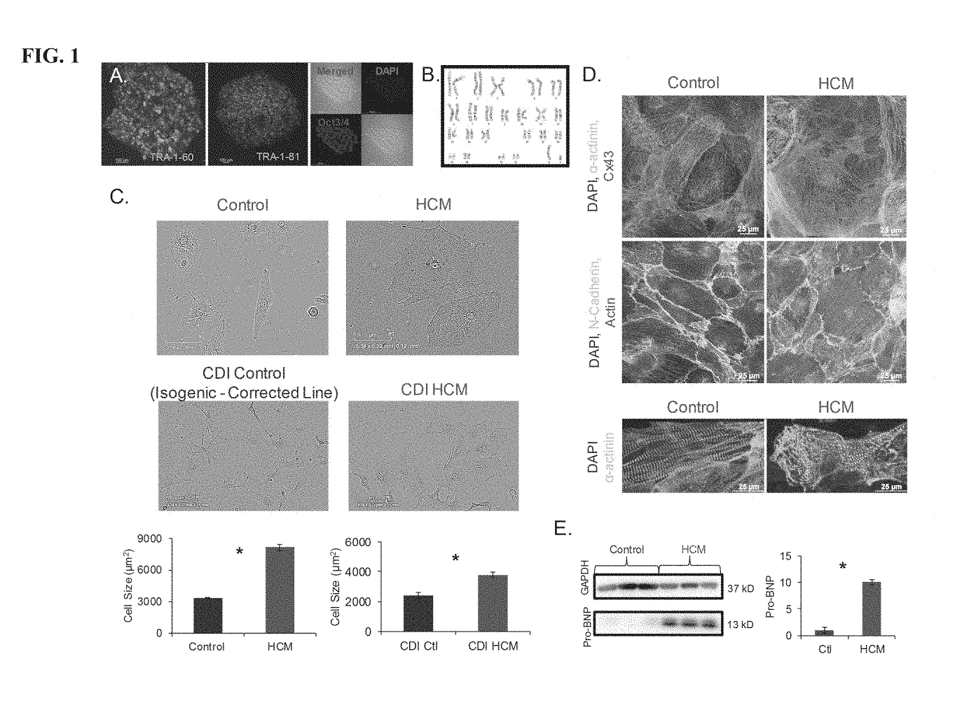

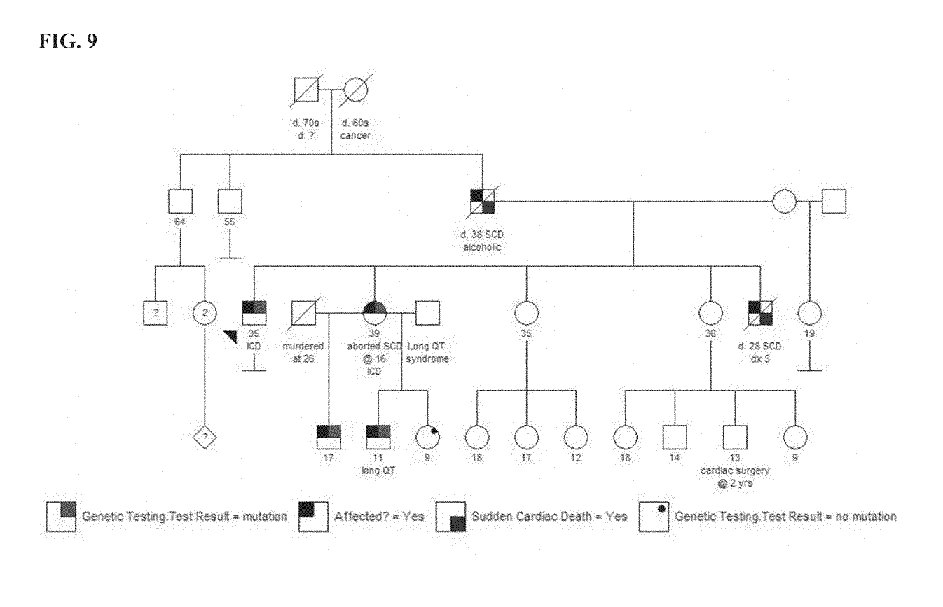

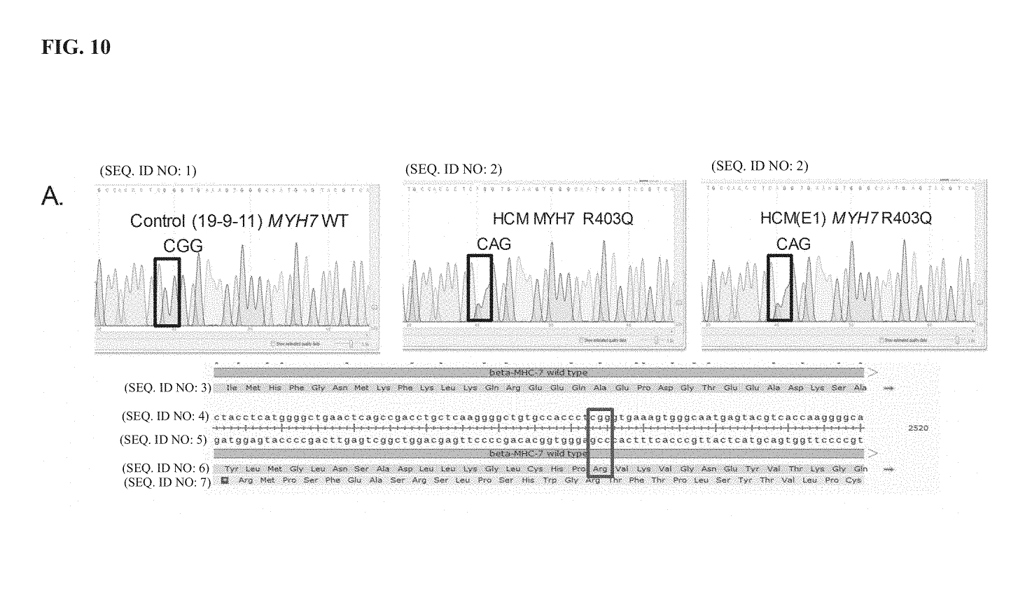

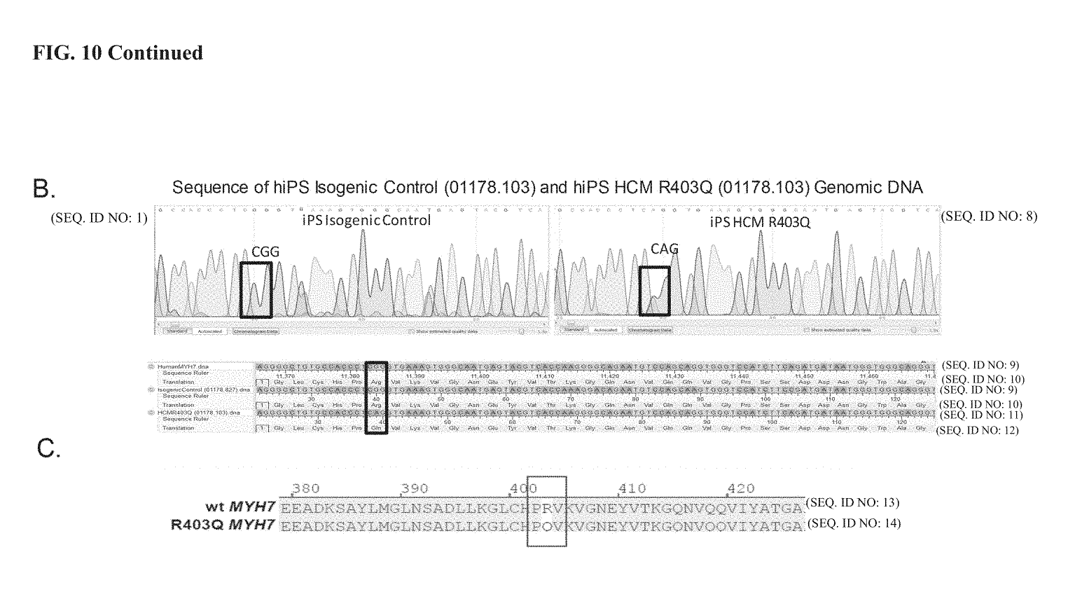

Dermal Fibroblast Reprogramming and hiPSC Maintenance—A skin biopsy was obtained from an HCM patient (referred to as HCM). See FIG. 9 for inheritance pattern and patient phenotype. Presence of the Arg403Gln mutation of human MYH7 was determined by genotype analysis (FIG. 10). Early passage fibroblasts were cryopreserved, and subsequently dermal fibroblasts were reprogrammed to hiPSCs using both a vector free approach by nucleofection of episomal reprogramming vectors as described previously (Yu J, et al., Science. 2009; 324:797-801). Pluripotency of HCM patient specific iPSCs was confirmed by immunofluorescence analysis of pluripotency markers, and normal chromosomal arrangement was confirmed by karyotype analysis (FIG. 1). hiPSCs were maintained on Matrigel using commercially available stem cell medium (mTeSR™ 1, Stemcell Technologies, StemMACS™ iPS-Brew XF). hiPSCs were maintained as colonies and passaged every 5-7 d (Bizy A, et al., Stem Cell Research. 2013; 11:1335-1347; Herron T J, et al., Circulation: Arrhythmia and Electrophysiology. 2016; 9). Two non-related hiPSC control lines were used: BJ-iPSC, generated using mRNA reprogramming (Bizy et al., supra); and the 19-9-11 cell line (obtained from WiCell), generated using episomal vectors (Yu et al., supra).

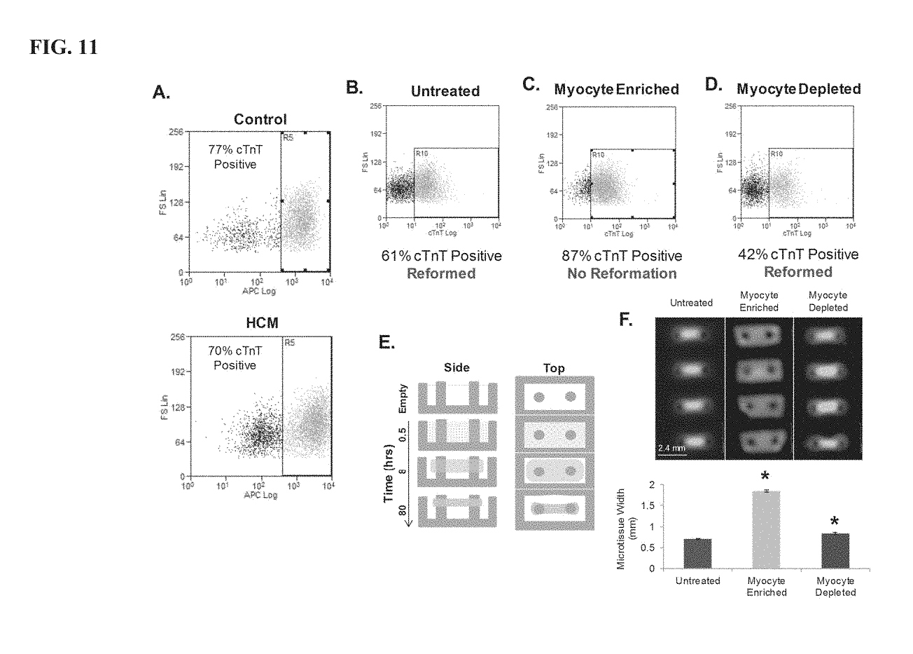

Additional hiPSCs were obtained from Cellular Dynamics International, one from an HCM patient (referred to as CDI HCM, MyCell® Products iPSC ID#: 01178.103) with the same mutation and the isogenic control for this patient (referred to as CDI Control, MyCell® Products Engineered iPSC CUS-iPSC-ENG ID#: 01178.827) where the Arg403Gln mutation of MYH7 was corrected with transcription activator-like effector nuclease (TALEN). Both the HCM mutation and correction were confirmed with sequencing (FIG. 11).

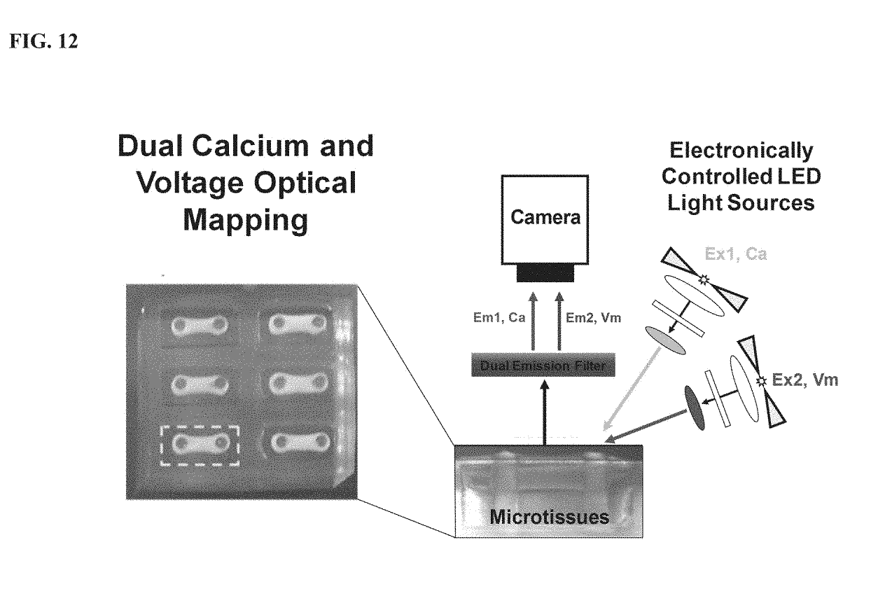

hiPSC and hiPSC-CM Culture—hiPSCs grown in the absence of feeder-cells were re-plated as monolayers for cardiac differentiation using small molecules as previously described (Lian X, et al., Nat Protoc. 2013; 8:162-175; Zhang J, et al., Circulation Research. 2009; 104:e30-e41). Only myocyte cultures with a completely confluent area of beating were used. These cultures typically had proportions of 74.5±4.1% myocytes (cTnT+) and 25.5±4.1% non-myocytes (cTnT−) (FIG. 12), which is a proportion consistent with previous reports for 3D microtissue optimization (Thavandiran N, et al., Proc Natl Acad Sci USA. 2013; 110:E4698-4707). HCM myocyte percentages did not differ significantly from controls (72.4±3.2% cTnT+ cells). Monolayers were plated on 40D polydimethylsiloxane (PDMS) silicone sheeting (SMI; Saginaw, Mich.) coated with Matrigel (Corning) at a density of 1×10⁶ cells/ml as described recently (Herron T J, et al., Circ Arrhythm Electrophysiol. 2016; 9).

For generating 3D microtissues a collagen solution of 50% EB20, 25% High Concentration Collagen Type 1 (9.06 mg/ml, Corning), 20% DI water, 5% 10× Phosphate Buffered Saline (Gibco) and 0.006% sterilized 1M NaOH was prepared before adding hiPSC-CMs at a density of 4×10⁶ cells/ml. The collagen solution containing cells was then added into PDMS molds and maintained at 37° C. and 5% CO₂ for 20 min until collagen was crosslinked. FIG. 2 shows 6 PDMS microtissue molds, each 2×4×1.5 mm with 2 PDMS posts 0.5 mm in diameter and 2 mm apart. EB20 was then added and used for cell culture for 4 days, after which they were transferred into RPMI/B27 (Gibco) for a total of 7-10 days before experimentation. In control experiments using acute gene transfer to express the Arg403Gln mutation in control hiPSC-CMs, cells were transduced with ad-WT-MYH7 or ad-Arg403Gln-MYH7 two days before microtissue formation with 50 multiplicity of infection (MOI) and treated again on the day of 3D tissue formation with 100 MOI recombinant adenovirus.

Polydimethylsiloxane (PDMS) Mold Manufacturing—The microtissue culturing mold was designed in Solidworks (Dassault Systèmes Solid Works Corporation) and was 3D printed by stereolithography (ProX 950, 3D Systems). PDMS replicas of the 3D printed mold were prepared by standard soft lithography, using a 10:1 (w/w) PDMS monomer to curing agent ratio (Dow Corning, FIG. 2).

Dual Calcium and Voltage Optical Mapping—Action potential duration (APD), calcium transient duration (CaTD) and arrhythmia characterization were studied as previously described (Lee P, et al., Circ Res. 2012; 110:1556-1563; Bollensdorff C, et al., European journal of physiology. 2011; 462:39-48). Calcium and voltage measurements conducted on the Nikon MR laser scanning confocal microscope (Nikon Instruments Inc, Melville, N.Y.) under the 10× objective. Calcium measurements were conducted with Fluo-4 (10 μM, Life Technologies) for and for voltage measurements FluoVolt (Fisher Scientific) was used following manufacture recommendations of 10 μl of Power Load and 1 μl of FluoVolt per ml of HBSS (in mmol/L:1.6 CaCl₂, 5.4 KCl, 0.8 MgSO₄, 0.4 KH₂PO₄, 4.2 NaHCO₃, 136.9 NaCl, 0.3 NaHPO₄, 5.5 D-Glucose, and 10 HEPES; pH 7.4, Thermo Fisher Scientific), the dye was then added to cells for 20 minutes and then changed to warmed HBSS for optical mapping. Tissues were kept at 37° C. for the duration of the experiment and movies were recorded at (60 frames/s, 6.27 μm/pix over 512×256 pixels).

Optical mapping, shown in the schematic in FIG. 12, was used for voltage recordings or dual calcium and voltage recording as previously described. FluoVolt (Fisher Scientific) was used as described above for voltage recording, while dual calcium and voltage recordings used dyes Rho-2-AM (10 μM, ThermoFisher Scientific) and di-4-ANBDQPQ (70 μM, AAT Bioquest).

Tissue Contractile Force Measurements—Contractile force was measured using the high speed resonant scanner of the Nikon MR confocal imaging system. Videos of contracting cardiac microtissues were collected (60 frames/s, 6.27 μm/pix over 512×256 pixels) and saved for post-acquisition analysis. Displacement of the posts was converted into contractile force F using Hooke's Law,

$$F = - \frac{3 {π Er}^{4}}{2 {a}^{2} ( 2 L - a )} x$$

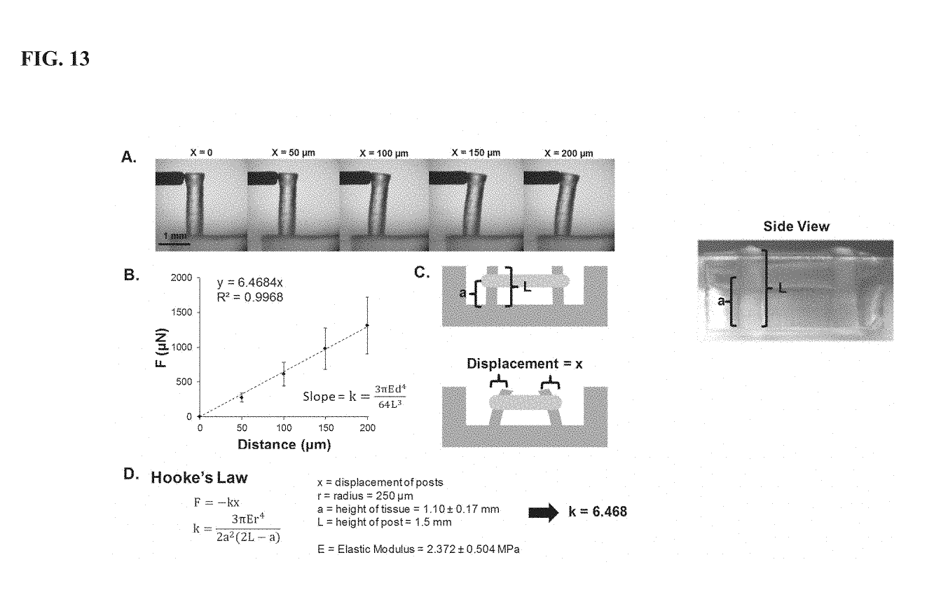

(Wang Y-1, Discher D E. Cell mechanics. Amsterdam; Boston: Elsevier Academic Press; 2007), where E is the elasticity modulus of PDMS and E=2.37±0.50 MPa (FIG. 13), r is the post radius and r=250 μm, a is the post height and a=1.5 mm, L is the height of tissue on the post and L=1.10±0.17 mm, and x is the maximal displacement for each contraction obtained from optical traces.

Flow Cytometry—Flow cytometry analysis was performed for comparison of percentages of cTnT positive and negative hiPSC-CMs. Briefly, cells were dissociated with 0.5% trypsin (Gibco) for 5 minutes and collected in EB20. Cells were centrifuged at 1000 RMP for 5 minutes and supernatant was removed. Cells were fixed in 200 μl of cold 3% PFA per 2×10⁵ cells for 10 minutes. Cells were rinsed with 1000 μl of cold PBS, centrifuged and re-suspended in 200 μl of cold permeabilization buffer and incubated on ice for 10 minutes. Centrifuge and re-suspend cells in 200 μl of blocking buffer and incubate for 15 minutes. Add 1 ml of cold 3% bovine serum albumin (BSA, Sigma) in permeablization buffer and centrifuge. Remove supernatant and add 50 μl of cold antibody and incubate for 40 minutes on ice. Add 50 ul of 2× DAPI (1:1000 dilution; Invitrogen) and incubate for 5 minutes and rinse with 1 ml of cold MACS buffer (Miltenyi Biotec). Re-suspend pellet in 1 ml of cold MACS Buffer for flow cytometry. Cells were stained as previously described (Kumar et al., supra), using DAPI and anti-cTnT-APC antibody (1:100, Miltenyi Biotec). Flow cytometry was conducted on a CyAN™ (Beckman Coulter) and analyzed in Summit 4.3 (Beckman Coulter) (Bizy et al., supra).

Mutagenesis and Recombinant Adenovirus Production—Details for the cloning and production of adenovirus to express the full length human β-MyHC motor protein have been reported previously (Herron T J, et al., Circulation Research. 2007; 100:1182-1190). The mutated amplimer sequence for the Arg403Gln missense mutation in the context of the human MYH7 gene was identified from the CardioGenomics data base (Genomics of Cardiovascular Development, Adaptation, and Remodeling. NHLBI Program for Genomic Applications, Harvard Medical School (www.cardiogenomics.org [accessed May, 2006]). The mutation is in exon 13 and the codon change is CGG>CAG. Site directed mutagenesis was performed by polymerase chain reaction using protocols of the Stratagene Quick Change mutagenesis kit to introduce the Arg403Gln mutation into the human MYH7 gene. Successful site directed mutagenesis was confirmed by DNA sequencing of the full length cDNA (University of Michigan DNA sequencing core) and translation of the wild-type and mutant myosin are shown in FIG. 102C. Homology is highlighted in yellow; the missense mutation is apparent at amino acid position 403 (white). The AdMax system (Microbix) was used to generate recombinant adenoviral vectors as previously described (Herron et al., 2007, supra; Davis J, et al., Circulation Research. 2007; 100:1494-1502; Rust E M. Molecular and cellular biochemistry. 1998; 181:143-155). Adult rat cardiac myocytes were isolated from female Sprague Dawley (Harlan, 150-200g) rats and cultured; gene transfer was performed as reported previously (Herron et al., 2007, supra). Rat cardiomyocytes exclusively express α-MyHC, thus providing a null background on which to express the human MYH7 gene as described before (Herron et al., 2007, supra). This enabled verification of the efficiency of gene transfer as well as the proper sarcomeric incorporation of the Ad-WT-MYH7 and Ad-Arg403Gln-MYH7 constructs. Calcium tolerant cardiac myocytes were plated on laminin coated coverslips (˜2×10⁴ myocytes/coverslip) and treated with Arg403Gln or WT adenovirus in M199 medium. MOI was 500 for each virus. Myocytes were maintained in primary culture in M199 medium for up to four days.

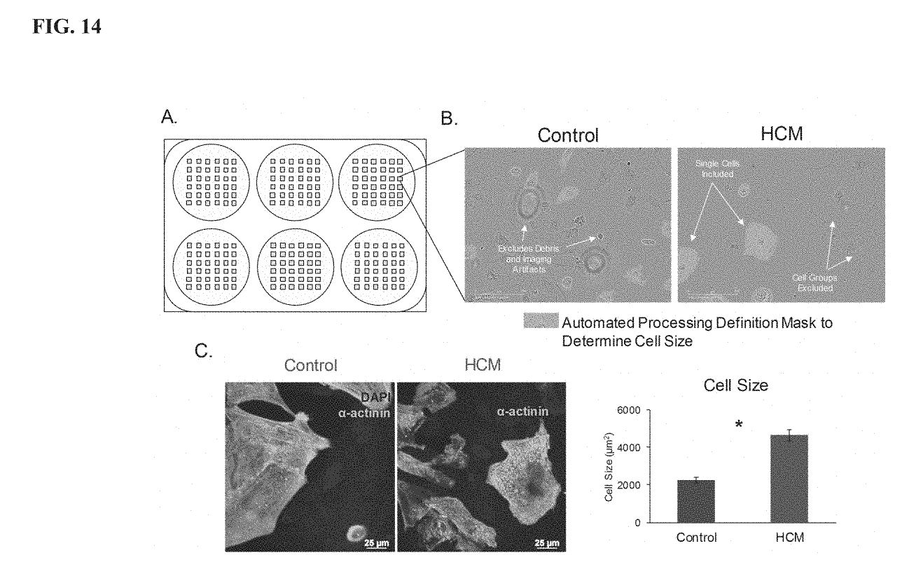

Cell Size Quantification—hiPSC-CMs were magnetically purified using the PSC-Derived Cardiomyocyte Isolation Kit (Miltenyi Biotec), myocytes were plated at a low density of 20,000 cells per 35 mm well on Matrigel (Corning) coated PDMS (SMI) and allowed to grow for 7 days. An IncuCyte® Zoom Live Cell Analysis System (Essen BioScience) was used to acquire images of live single cells with a 20× objective for high content analysis of hiPSC-CM size. An automated processing definition was determined for each cell line optimized to include analysis exclusively of single cells. 20 images from each group were randomly selected to assess fidelity of the processing definition; maximal and minimal selection criteria were reduced or increased based on the sample of images to exclude debris and cell clusters. The processing definition was then applied to all images. FIG. 14A-B demonstrates the approach for automated cell size quantification. Cell size was also measured manually using immunostained hiPSC-CMs plated as monolayers (FIG. 14C).

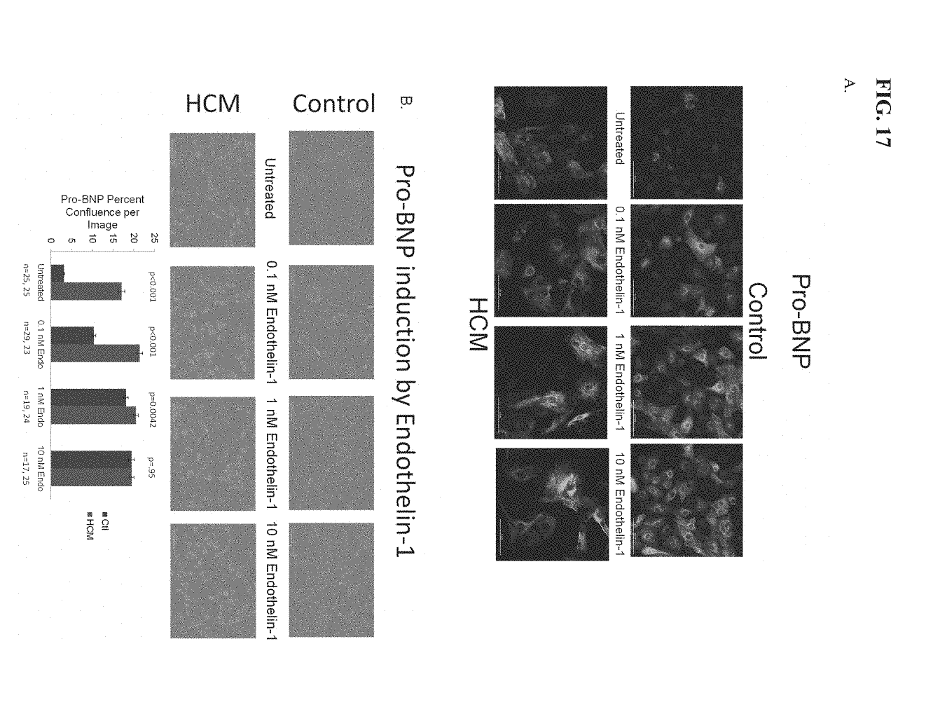

Endothelin-1 Treatment—Magnetically purified myocytes were plated at 50,000 cells per 96 well in EB20, on day three of culture cells were transferred to B27/RPMI. On day 4 cells were treated with either fresh B27/RPMI or B27/RPMI with Endothelin-1 (ET-1, Sigma) ranging from 0.1 nM to 10 nM. In order to retain the brain naturetic peptide (BNP) within the cells, after 15 hours, monolayers were additionally treated with 10 μg/ml of Brefeldin A (Sigma). Brefeldin A prevents the excretion of BNP by inhibiting the function of GBF-1, a guanine nucleotide exchange factor, by binding with Arflp and preventing the conversion to the GTP-bound form, the lack of active Arflp prevents coat protein recruitment leading to a fusion of the ER and Golgi membranes due to the lack of vesicle formation. At hour 18 of ET-1 incubation, cells were fixed for staining or collected for western blots (FIGS. 14-18) (Carlson C, et al., Journal of biomolecular screening. 2013; 18:1203-1211).

Immunofluorescence and Western Blots—Cardiomyocytes were fixed for immunocytochemistry and western blots as previously described (Herron et al., 2002, supra; Herron et al., 2016, supra; Bizy A, et al., Stem Cell Res. 2013; 11:1335-1347). For immunostaining, cells were washed with phosphate buffered saline (PBS, Corning), fixed with 3% paraformaldehyde (Electron Microscopy Sciences) for 10 minutes and rinsed twice before blocking with 5% donkey serum (EMD Millipore) in PBS (Corning) plus 0.1% Triton X-100 (Fisher) for 1.5 hours at room temperature. Staining done for pro-BNP in endothelin-1 experiments instead used 3% (w/v) nonfat dry milk (Great Value™) in 0.1% (v/v) Triton X-100 (Fisher) in PBS (Corning) for blocking and antibody preparation. Primary antibodies include α-actinin (1:500, Sigma), Cx43 (1:100, Millipore), β-MyHC (1:200, ATCC clone A4.951), actin (1:300, Sigma), pro-Brain Naturetic Peptide (pro-BNP, 1:500, Abcam, 13115), N-cadherin (1:200, BD Bioscience) were added to 5% donkey serum in PBS plus 0.1% Triton X-100 PBS or 3% NFM in 0.1% Triton X-100 PBS and incubated overnight at 4° C. with constant agitation. Subsequently, samples were washed three times in 0.1% Triton X-100 PBS. Secondary antibodies AlexaFluor® 488 (1:1000, Life Technologies) or AlexaFluor® 594 (1:1000, Life Technologies) were diluted in the same solution as primary antibodies and incubated at room temperature in the dark for 1.5 hours. Samples were then washed three times with 0.1% Triton X-100 PBS and once with PBS. Finally, nuclei were stained with 4′,6-diamindino-2-phenylindole (DAPI, 1:1000 dilution; Invitrogen) for 10 minutes at room temperature in the dark. Some samples were first kept in PBS were evaluated with a IncuCyte° Zoom Live Cell Analysis System (Essen BioScience, Ann Arbor, Mich.) with either a 4× or 20× objective before being mounted using Fluoromount-G® (Southern Biotech) on microscope slides and coverslips (Fisher Scientific) and evaluated using a Nikon MR laser scanning confocal microscope and software (Nikon Instruments Inc, Melville, N.Y.) using 10×, 20× or 40× and 60 oil immersion objectives (Nikon).

Samples for western blots were collected in laemmli sample buffer (BioRad Laboratories) and 0.05% β-Mercaptoethanol (Sigma) and stored at −20° C. Samples were run on 15 well NuPage™ 4-12% Bis-Tris glycine gels (Invitrogen) using a Power Ease 500 electrophoresis power supply (Invitrogen) run at 80 V, 120 mA and 25 W in NuPage® MOPS SDS Running Buffer (Life Technologies). A Precision+Protein™ Dual Color Standard (BioRad Laboratories) was used as a marker. Proteins were transferred to a nitrocellulose membrane (BioRad Laboratories) between 6 pieces of gel dry transfer paper (BioRad Laboratories) on using a semi-dry transfer unit (TE77XP, Hoefer) run at 90 mA with NuPage® Transfer Buffer (Life Technologies). Phosphate buffered saline (PBS, Corning) along with Tween® 20 (Fisher) was used to make a 0.05% Tween20 PBS-T solution and blocking buffer was made with 5% nonfat instant dry milk (Great Value™) in PBS-T.

Antibodies used for western blots include pro-Brain Naturetic Peptide (pro-BNP, 1:250, Abcam, 13115), β-MyHC (1:1000, ATCC clone A4.951), GAPDH (1:1000, Sigma, G8795) and horseradish peroxidase (HRP) conjugated secondary antibody (1:1000, Jackson Immunoreasearch). Blots were treated with Pierce ECL Western Blotting Substrate (Thermo Scientific) and imaged with a Gel Dock System (BioRad Laboratories). Processing was done in Image Lab 5.2.1 build 11 (BioRad Laboratories), samples were background subtracted, normalized to a loading control either GAPDH and then normalized to the control or untreated condition.

Statistics—One way ANOVA with Bonferroni's multiple comparisons test and Student's t-test are used when appropriate. P-values<0.05 are considered statistically significant.

### Results

3D Cardiac Microtissues

hiPSC-CM 3D Self-Assembly—To develop a human, in vitro model of HCM which recapitulates not only the cellular hypertrophy but 3D structural phenotype of the disease, a 2×4 mm dual flexible cantilever system was used (Thavandiran et al., supra; Boudou T, et al., Tissue Eng Part A. 2012; 18:910-919) (FIGS. 2 & 3). 3D tissue formation does not adequately occur when using purified hiPSC-CMs (FIG. 11). However, by using differentiations with percentages of about 75% myocytes 3D microtissue capable of electrophysiological and contractile evaluation were generated.

Beginning with the self-directed formation kinetics of control hiPSC-CM tissues, a rapid formation was observed. Tissues reduced to 22.3±2.2% of their initial volume by 86 hrs, with an exponential time constant (r) of only 8.7 hrs (n=39, FIG. 4). Microtissues typically began to spontaneously beat around day 4 after plating, and at the time of experiments on day 7 they had an average beating frequency of 0.60±0.04 Hz.

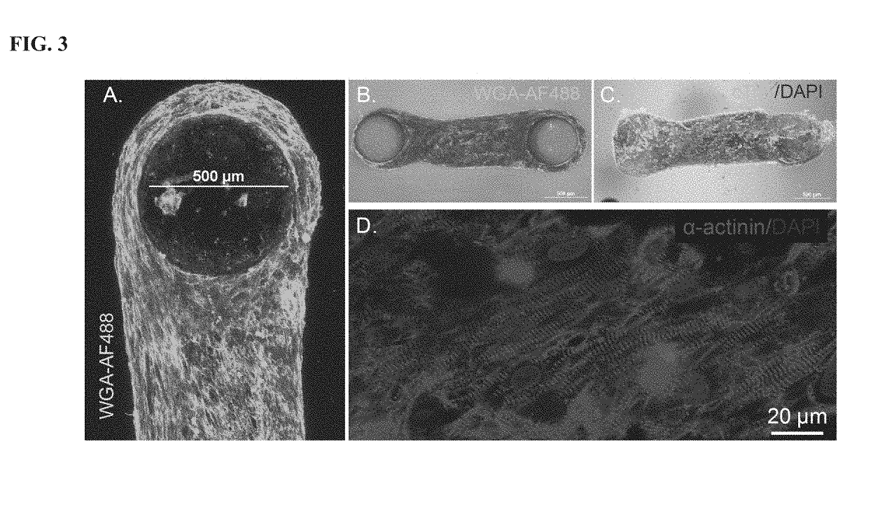

3D Structural Analysis—3D microtissues generate a superior structural organization compared with 2D preparations Boudou et al., supra; Hinson J T, et al., Science. 2015; 349:982-986; Mannhardt I, et al., Human engineered heart tissue: Analysis of contractile force. Stem Cell Reports). In FIG. 3A-B, cell membrane staining with WGA-AF488 shows a predominantly elongated structural organization and alignment around the PDMS posts throughout control 3D microtissues. FIG. 3C show homogeneous distribution of cardiomyocytes (cTnT+). This is mirrored by immunostaining for α-actinin revealing clearly aligned sarcomeres from myocyte to myocyte and with DAPI nuclei elongation (FIG. 3D). Microtissues elongate and align the sarcomere structure as well as the cardiomyocytes themselves.

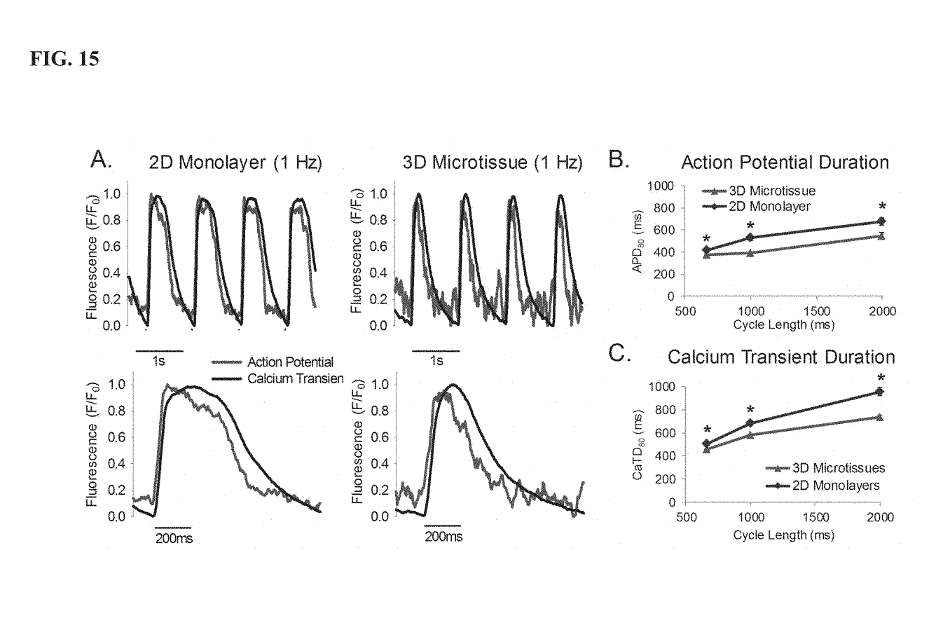

Functional Analysis of Control hiPSC-CM in 2D vs 3D—To assess the level of electrophysiological maturation of 3D microtissues, dual calcium and voltage optical mapping was used to quantify the action potential duration (APD) and calcium transient duration (CaTD) (Lee P, et al., Circ Res. 2012; 110: 1556-1563). 3D microtissues show significantly shortened action potential durations (APD₈₀ at 1 Hz: 526.8±22.2 ms vs. 395.0±8.7 ms, p<0.05) and calcium transient durations (CaTD₈₀ at 1 Hz: 580.9±8.6 ms vs. 681.8±15.2 ms, p<0.05, FIG. 15) compared with matched 2D monolayers. The shortened APDs in 3D tissues approaches the values observed in optically mapped human ventricular preparations (APD₈₀ at 1 Hz: 383-494 ms (Boukens B J, et al., Cardiovascular research. 2015; 108:188-196) or 317-360 ms (Glukhov A V, et al., Circ Res. 2010; 106:981-991). These findings indicate there is a significant level of maturation in the membrane ion channel densities and excitation-contraction coupling machinery in 3D hiPSC-CM microtissues. In some embodiments, APD₈₀ at 1 Hz is 500 ms or less, 450 ms or less, or 400 ms or less.

To determine whether this electrophysiologic maturation was due to the tissue formation alone, or due to the tissue formation in the context of a tensile load between microposts, 3D microtissues were generated without posts. For these unstructured 3D tissues APD at 1 Hz is comparable to 2D preparations (526.8±22.2 ms vs. 518.4±20.9 ms, n.s.); both being significantly longer than the structured 3D 2-post preparations (395.0±8.7 ms, p<0.05). This finding suggests that the mechanotransductive force exerted by the tissue on the flexible PDMS posts acts as a mechanical cue to induce structural myocyte alignment and maturation, possibly through a mechanism similar to in vivo cardiac development in the presence of afterload.

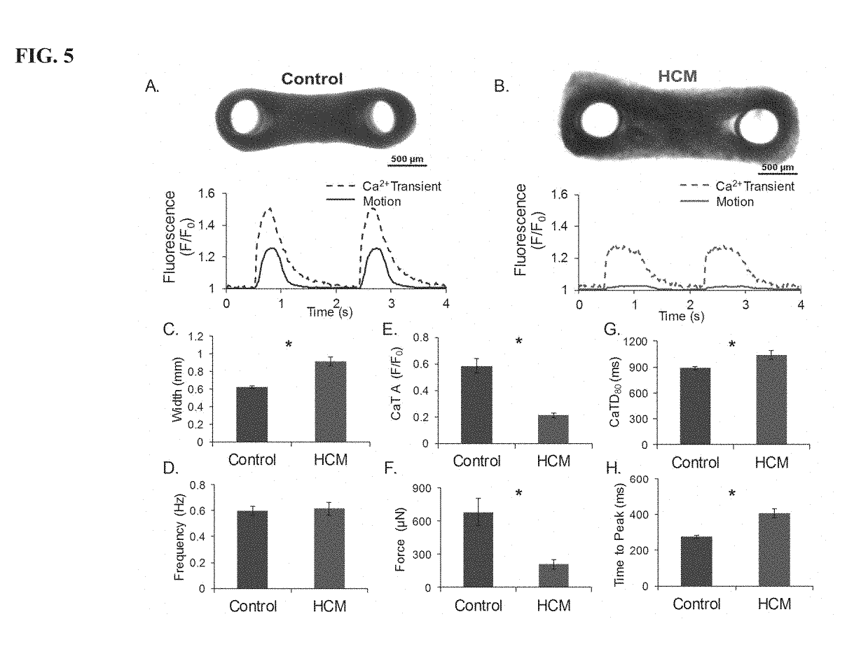

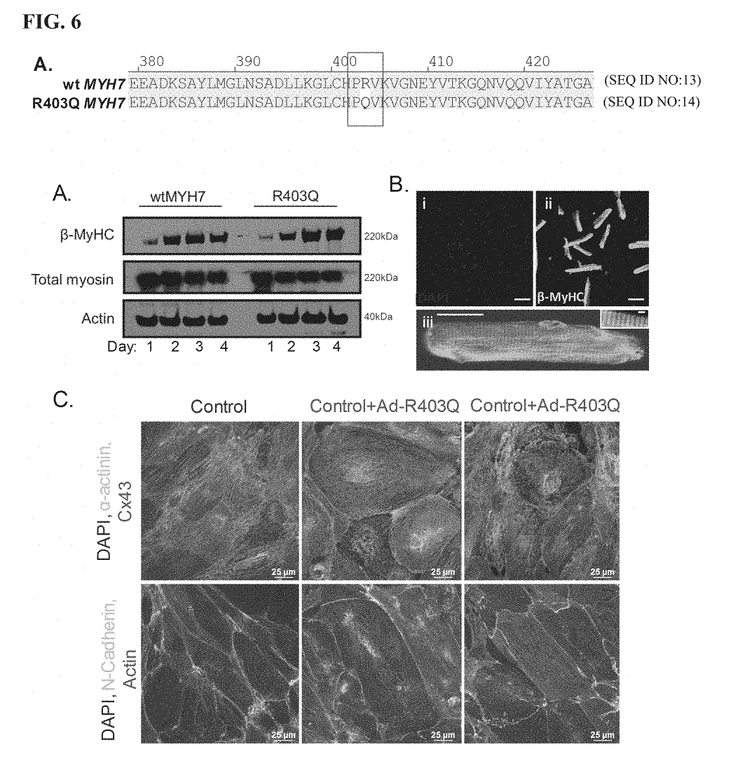

A major advantage of the 3D microtissues over 2D monolayers is that they can be used to evaluate force of contraction. FIG. 5 show contraction, force and CaT in a 3D microtissue. With the 2-post model, post displacements of 64.1±11.5 μm and contractile forces of 680.1±122.4 μN or 2.39±0.43 mN/mm² when normalized to cross-sectional area (n=38) were measured. This is approximately 4-fold greater than in recent reports using smaller 3D microtissue models which generated forces of ˜0.5 mN/mm² (Boudou et al., supra; Hinson J T, et al., Science. 2015; 349:982-986). Additionally, although the tissue contractile force generated here is less than what is reported for adult cardiac tissue (Rossman E I, et al., J Mol Cell Cardiol. 2004; 36:33-42) (20-45 mN/mm²), it is greater than the 1.1-1.7 mN/mm² reported for infants (Wiegerinck R F, et al., Pediatric research. 2009; 65:414-419). In some embodiments, tissue contractile force is greater than 1.0, 1.5, 1.8, 2.0, or 2.5 mN/mm².

3D Cardiac Microtissues

A Human Model of Hypertrophic Cardiomyopathy HCM hiPSC-CM Model—3D microtissues made from control cells generate a reproducible formation profile, with improved cellular structure and organization, maturation and contractile forces. With this model, it was investigated whether 3D microtissues can be used to distinguish the hallmark structural features and characteristics of inherited HCM. To this end, hiPSC-CMs were generated from a skin biopsy of a patient with HCM with an Arg403Gln mutation in MYH7 which codes for β-myosin heavy chain (Geisterfer-Lowrance et al., supra) (FIGS. 1, 9, and 10). This patient was diagnosed at 28 years of age with a strong family of history of sudden cardiac death. His phenotype includes both atrial and ventricular arrhythmias, severe diastolic dysfunction and a hypertrophied septum of 22 mm.

In 2D, the HCM hiPSC-CMs made from this patient are able to form functional monolayers exhibiting cardiomyocyte hypertrophy. Low density purified HCM hiPSC-CMs showed significant hypertrophy in comparison to controls (3339.8±35.1 μm² vs. 84614.1±318.3 μm², n=702, 1219, p<0.05, FIG. 1C), as did unpurified HCM hiPSC-CMs plated as monolayers (2268.7±147.6 μm² vs. 4657.6±308.0 μm², n=67, 90, p<0.05, FIG. 14C).

Additionally, an hiPSC line was obtained from Cellular Dynamics International made from a second patient with the same HCM mutation (CDI HCM) along with its isogenic control (CDI Control), where the Arg403Gln mutation had been corrected with TALEN. Although the HCM myocytes were not as large as those from the first HCM patient, they were still nearly double the size of their isogenic control line (2417.2±193.6 μm² vs. 3797.1±181.4 μm², n=97, 393, p<0.05, FIG. 1C).

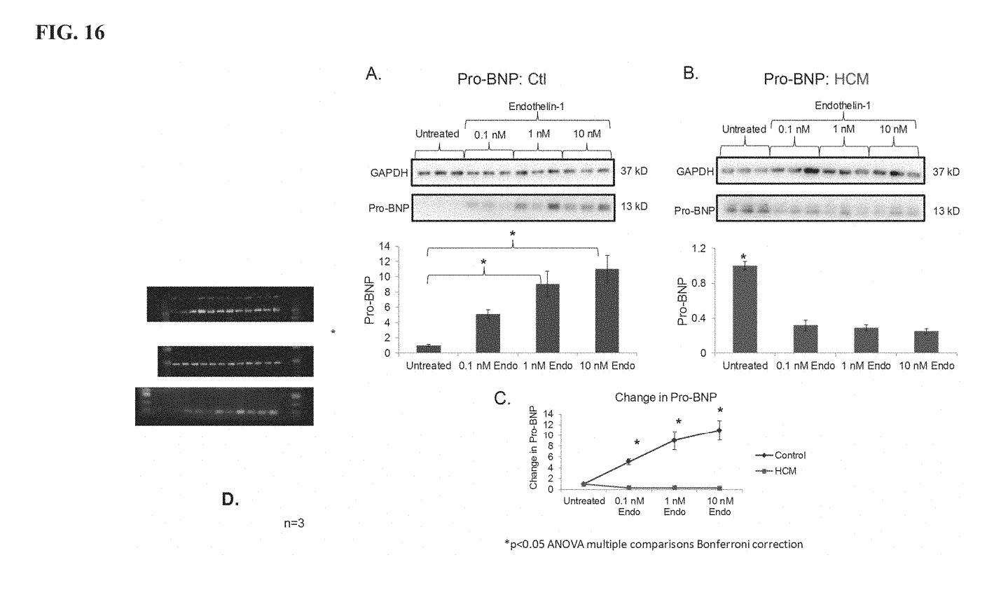

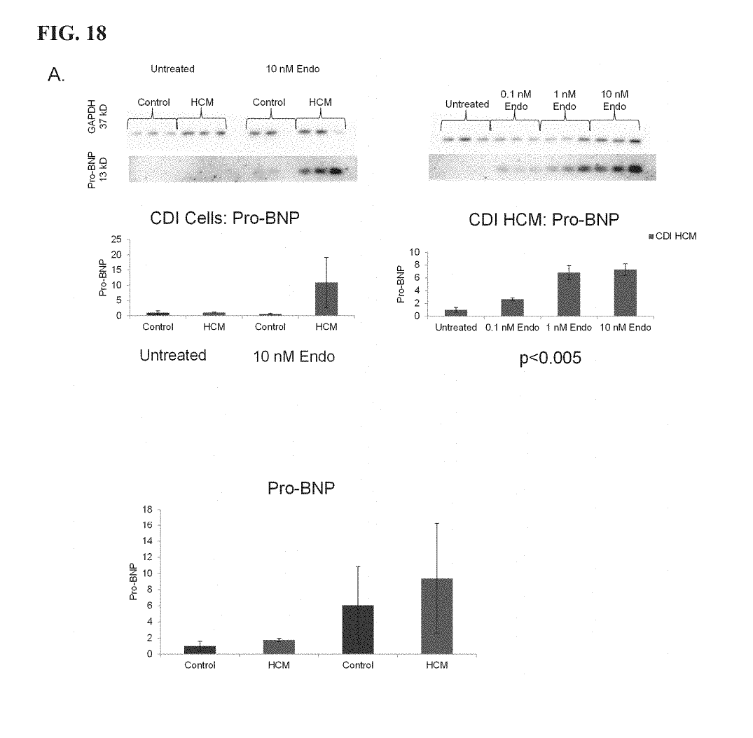

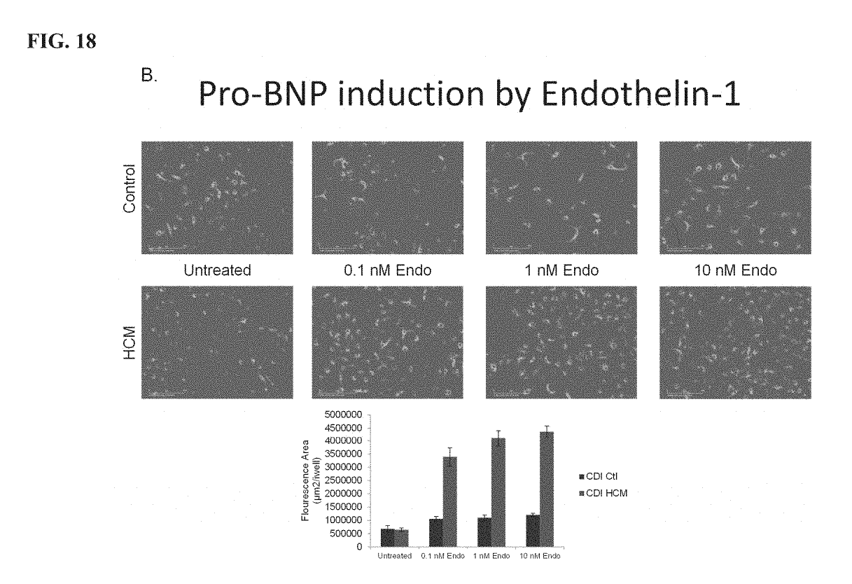

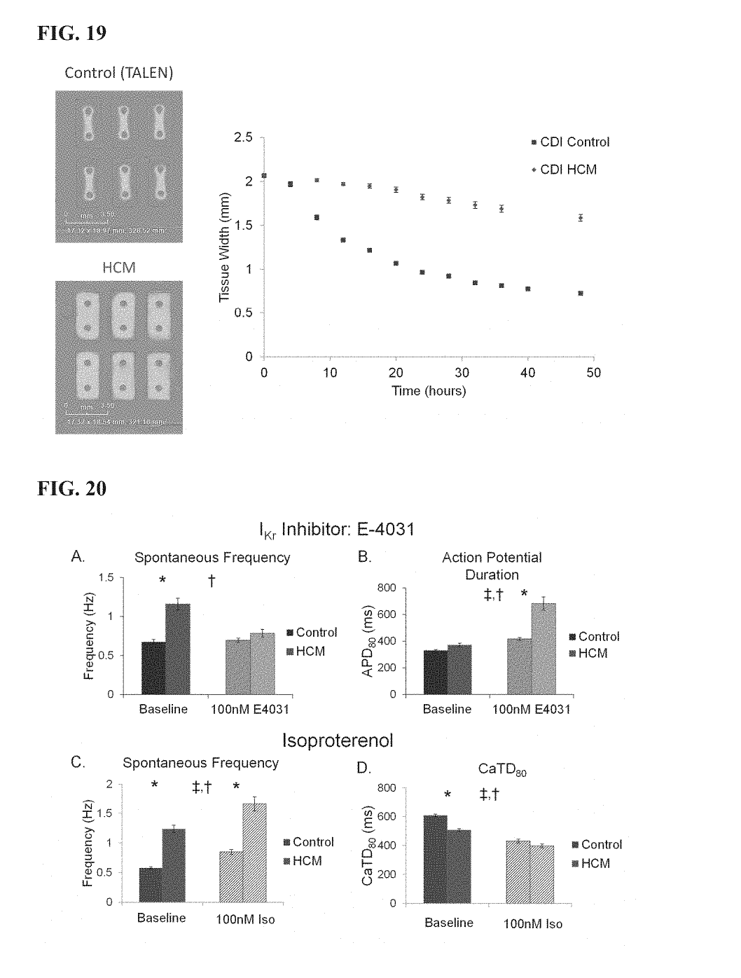

Purified myocytes from the first HCM patient exhibited higher levels of hypertrophic marker pro-brain naturetic peptide (pro-BNP, FIG. 1E). Moreover, when these cells were stimulated with endothelin-1 (ET-1) pro-BNP levels did not increase; indicating that the pathway was already maximally active. In control cells however, treatment with ET-1 resulted in a dose response increase in pro-BNP (FIG. 16). However, in both the CDI HCM and isogenic control myocytes, low baseline levels of pro-BNP were observed despite the significant hypertrophy observed in the HCM myocytes. Their response to ET-1 was also different; treatment resulted in a dose response for both lines with a much greater maximal response in the CDI HCM myocytes, indicating its pathway was not initially maximally active as observed in the other HCM patient, but that its hypertrophic response was more responsive to stimulation (FIG. 18). These results indicate that there is significant hypertrophic variability between patients which was mirrored by differences in expression of hypertrophic marker pro-BNP and its responsiveness to ET-1 stimulation.

3D Self-Assembly of HCM Cardiac Tissues—Significant structural disorganization is a distinguishing hallmark of HCM (Geisterfer-Lowrance et al., supra; Lopes et al., supra), which cannot be replicated with traditional 2D culturing techniques. Using this HCM patient specific bioengineered 3D cardiac microtissue model recapitulates this key feature.

In 2D, HCM monolayers form with no noticeable defects concerning how the cells organize themselves in culture. In 3D however, HCM cardiac tissues demonstrate marked organizational malformation and structural hypertrophy (FIGS. 4 & 5).

3D HCM tissues had a notably slowed rate of organization of the collagen matrix; by 86 hours the HCM microtissues were nearly twice the size of the control tissues, reducing to only 38.0±4.1% of their original size vs. 22.3±2.2% for controls (p<0.05). This difference remained after a week of growth, where the 3D HCM microtissues remained about twice the size 2.89±0.31 mm² vs. 1.70±0.17 mm² (p<0.05, FIG. 5C). Additionally, the rate of formation was significantly altered, HCM tissues took about 6 times longer to reach a steady state of formation: r=57.3 hours vs. 8.7 hours in controls (FIG. 4A).

HCM 3D formation deficits were also clearly seen in HCM tissues from our second patient line (HCM CDI, FIG. 4B). HCM tissues took nearly twice as long to reach steady state (r=32 hours vs. 16.5 hours) and the widths of the HCM tissues were more than twice the size of the isogenic control tissues (1.59±0.04 mm compared to 0.73±0.02 mm, p<0.05). We found that the patient's isogenic control line reformed in a similar manner to our other control lines, essentially preventing the HCM structural phenotype (19-9-11: 0.66±0.01 mm, BJ-iPSC: 0.79±0.01 mm).

With the 3D microtissue platform it is possible to recapitulate the structural disorganization and defining features of HCM. 3D culturing revealed that this mutation alters the self-assembled organizational kinetics demonstrating a marked inability to create the organized compacted tissue seen in controls.

HCM 3D Microtissue Structure & Function—HCM is characterized by both intracellular sarcomere disarray and, at the tissue level, intercellular disorganization. Both of these characteristics were observed in the HCM microtissues (FIG. 4C). At the cellular level, sarcomere banding patterns were often irregular in HCM hiPSC-CMs. While on the tissue level, we observed that HCM hiPSC-CMs were highly disorganized, and in comparison to controls they had less elongation, alignment and were in worse contact with one another (FIG. 4C).

Similarly to the HCM patient phenotype, these structural abnormalities were associated with electro-mechanical dysfunction. While spontaneous beating rates were similar between controls and HCM microtissues (0.60±0.04 vs. 0.62±0.05 Hz, n=38, 22, p=0.85), the HCM microtissues showed a significant CaTD₈₀ prolongation of 1042.0±53.1 vs. 881.9±21.2 ms (n=24, 38, p<0.05) and a significant decrease in the CaT Amplitude (CaTA) 0.21±0.02 vs. 0.60±0.05 F/F₀ (n=24, 38, p<0.05). This indicates abnormal calcium handling in the HCM microtissues (FIG. 5).

Additionally, 3D HCM microtissues responded to exposure to isoproterenol (I_{SO}) and I_{Kr} blocker E-4031 which respectively resulted in an increase in spontaneous frequency (0.58±0.02 Hz to 0.85±0.04 Hz, n=8) and an increase in APD₈₀ (326.2±1.2 ms to 405.9±13.6 ms, n=19, FIG. 20). Although HCM tissues responded normally to I_{SO}, their response to E-4031 was characterized by a more than 2-fold increase of the APD₈₀ (397.2±8.6 ms to 806.2±47.2 ms, n=19, p<0.05), far surpassing the APD₈₀ of the controls treated with E-4031 (405.9±13.6 ms vs. 806.2±47.2 ms, n=19, 12, p<0.05). From this it was determined that the repolarization of HCM tissues has a greater dependence on I_{Kr} and less of a dependence on I_{K1} than control tissues. As I_{K1} is the major current responsible for repolarization in adult cardiomyocytes this indicates that the HCM tissues stronger dependence on I_{Kr} may be an indication of impaired maturation. This further indicates that HCM 3D tissues are not able to obtain the full benefits of the mechanotransductive maturation conferred by 3D plating seen in control tissues.

Force Generation of HCM 3D Microtissues—Examination of the contractile forces showed that the 3D HCM tissues are significantly less than the forces generated by control tissues; 680.1±122.4 μN vs. 209.5±44.5 μN (n=38, 25, p<0.05). Control tissues, seen in Movies 3 and 4, clearly displace the flexible posts along their aligned axis (FIG. 5). However, HCM tissues only move the posts a third of that distance, despite having formed a functional syncytium with calcium transients observed synchronously throughout each tissue during contraction.

While all of the control tissues could displace the posts, 20% of HCM tissues were unable to generate enough coordinated directional force to bend the flexible posts with any measurable displacement and 48% of the tissues displaced the posts less than 10 μm. This demonstrates that the HCM tissues have a significant contractile dysfunction on a tissue level and indicates an inefficiency of their excitation contraction coupling.

Adenoviral Mediated Gene Transfer of HCM Arg403Gln Mutation—To ensure causality of the Arg403Gln mutation of our HCM phenotype we utilized adenoviral gene transfer to express the Arg403Gln MYH7 mutation in control hiPSC-CMs. Site directed mutagenesis was conducted, and the introduction of Arg403Gln in the human MYH7 gene was confirmed by sequence analysis (FIG. 10). Efficiency of viral gene transfer was quantified using adult rat cardiomyocytes as ˜98% for the mutant Arg403Gln as well as for WT-MYH7, comparable to values previously reported (Herron T J, Circulation Research. 2007; 100:1182-1190). Importantly, the Arg403Gln mutant protein localizes to the sarcomere in structurally normal adult rat cardiomyocytes (FIG. 6B).

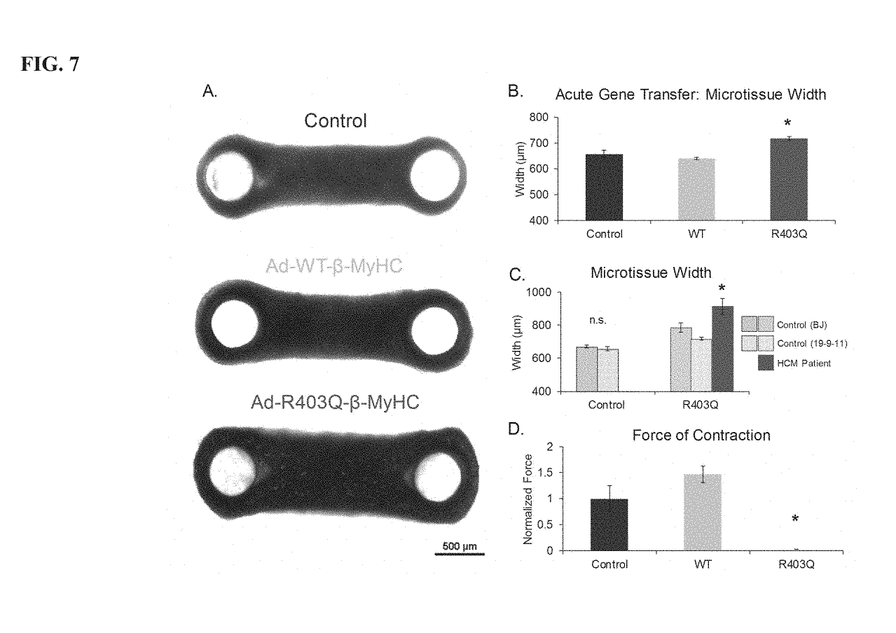

In 2D, control hiPSC-CMs driven to express the HCM Arg403Gln MYH7 gene showed the same cellular hypertrophy and sarcomere disorganization as seen in the HCM myocytes (FIG. 6C). Seven days post infection and microtissue formation, untreated controls and WT MYH7 expressing tissues had organized to a similar width of about 0.65 mm, while the Arg403Gln expressing tissues were about 50 μm wider (FIG. 7). Arg403Gln expression in both control lines yielded similar hypertrophy, while WT MYH7 infected lines remained similar to the untreated controls.

Although the degree of hypertrophy for the Arg403Gln expressing tissues was less than the HCM patient derived microtissues, the fact that even brief expression of Arg403Gln-mutant MYH7 in control myocytes was able to create tissue hypertrophy similar to the HCM patient derived tissues confirms that this finding is specifically driven by the Arg403Gln mutation, and not due to stem cell line variability.

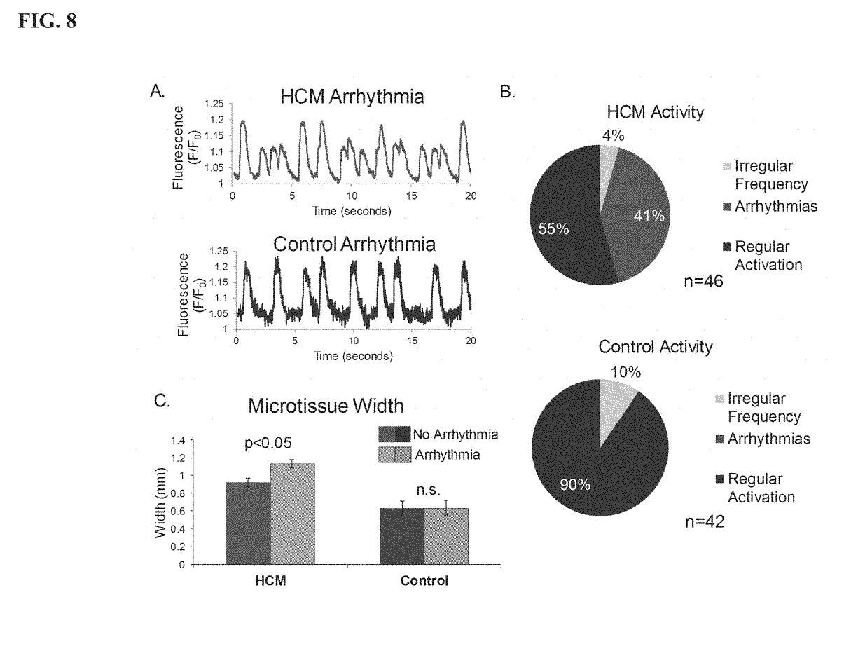

Additionally, it was found that Arg403Gln expression essentially eliminated the tissues ability to generate a contractile force capable of displacing the posts during contraction, while ad-WT-MYH7 treated cells displayed normal slightly increased contractile force (FIG. 8D). Taken together, these data confirm that the MYH7 Arg403Gln mutation plays a causal role in the structural and functional derangements observed in the patient derived microtissues.

HCM and Arrhythmias—In addition to recapitulating HCM hallmarks of structural disorganization and ineffectual contraction it was found that the 3D HCM tissues recapitulated the arrhythmogenic phenotype as well. 3D control microtissues exhibited spontaneous beating originating from a dominant pacemaker site resulting in homogenous and regular activation of the tissue 90% of the time (n=42, FIG. 8). In contrast, 3D HCM microtissues recapitulate the arrhythmia prone electrical phenotype of HCM patients, having spontaneous arrhythmias 45% of the time (n=46, FIG. 6).

All of the arrhythmias observed in control tissues were mild and limited to cycle length variability where the synchronous pattern of activation throughout the tissues remained intact (FIG. 8). By comparison, HCM microtissues presented with two kinds of arrhythmias; 10% were similar to those seen in controls, while 91% of HCM arrhythmias consisted of irregular patterns of fibrillatory conduction. Often, disjointed and irregular spatial and temporal propagation was observed throughout the HCM microtissues with intermittent reentry around the posts (FIG. 8); which is consistent with the HCM structural and electrical phenotype.

In HCM tissues HCM organizational deficits correlated with electrical behavior. The larger and less organized the microtissue the greater its propensity for arrhythmias (FIG. 8). Notably, an association between formation size and arrhythmia propensity was not observed in control tissues. Altogether these results demonstrate that 3D hiPSC-CM microtissues recapitulate the hypertrophic and arrhythmogenic phenotype caused by the HCM Arg403Gln mutation.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method of generating a three dimensional cardiac microtissue, comprising:
a) differentiating inducing pluripotent stem cells (iPSCs) to a population of cells comprising cardiac myocytes;
b) contacting said population of cardiac myocytes with a collagen solution; and
c) transferring said collagen solution comprising cardiac myocytes into a solid support and culturing under conditions such that said myocytes form a three-dimensional cardiac microtissue.

2. The method of claim 1, wherein said solid support comprises a plurality of microtissue culturing molds each comprising a plurality of posts spaced at a uniform distance.

3. The method of claim 1, wherein each of said microtissue molds comprises two posts.

4. The method of claim 3, wherein said posts are 1 to 3 mm apart.

5. The method of claim 4, wherein said posts are 2 mm apart.

6. The method of claim 2, wherein said posts are 0.2 to 1 mm in diameter.

7. The method of claim 2, wherein said microtissue molds are approximately 2×4×1.5 mm.

8. The method of claim 2, wherein said microtissue molds and said posts are made of polydimethylsiloxane (PDMS).

9. The method of claim 2, wherein said three-dimensional microtissue forms suspended between said posts.

10. The method of claim 1, wherein said iPSCs comprise a Arg403Gln mutation in the MYH7 gene.

11. The method of claim 10, wherein said three-dimensional microtissue exhibit one or more properties of hypertrophic cardiomyopathy (HCM).

12. The method of claim 11, wherein said properties of HCM are selected from the group consisting of structural disorganization, intracellular sarcomere disarray, reduced contractile force, arrhythmia, and electro-mechanical dysfunction.

13. The method of claim 1, wherein said population of cells comprises approximately 75% cardiac myocytes.

14. The method of claim 1, wherein said three dimensional cardiac microtissue exhibits beats.

15. The method of claim 1, wherein said three dimensional cardiac microtissue exhibits one or more parameters selected from the group consisting of electrophysiological maturation and contraction.

16. A three dimensional cardiac microtissue generated by the method of claim 1.

17. The three dimensional cardiac microtissue of claim 16, wherein said microtissue is suspended between two posts of a solid support comprising a plurality of microtissue culturing molds each comprising a plurality of posts spaced at a uniform distance.

18. A system, comprising:
a) a population of cells comprising cardiac myocytes; and
b) a solid support comprising a plurality of microtissue culturing molds each comprising a plurality of posts spaced at a uniform distance.

19. The system of claim 18, wherein said system further comprises one or more test compounds.
